(19) United States
(12) Patent Application Publication
(10) Pub. No.: US 2014/0271465 A1
(43) Pub. Date: Sep. 18, 2014

(54) MATRIX METALLOPROTEASE (MMP) TARGETED AGENTS FOR IMAGING AND THERAPY

(71) Applicant: Mallinckrodt LLC, Hazelwood, MO (US)

(72) Inventors: John N. Freskos, Clayton, MO (US); Mary Dyszlewski, Creve Coeur, MO (US); Lori Chinen, St. Charles, MO (US); Hector Knight, St. Louis, MO (US); Michelle Schmidt, Millstadt, IL (US)

(73) Assignee: Mallinckrodt LLC, Hazelwood, MO (US)

(21) Appl. No.: 14/208,350

(22) Filed: Mar. 13, 2014

Related U.S. Application Data
(60) Provisional application No. 61/791,387, filed on Mar. 15, 2013.

(51) Int. Cl. A61K 51/04; A61K 45/06; A61K 31/4545; C07D 211/66; C07D 401/12; A61K 31/4523

(52) U.S. Cl. CPC A61K 51/0482; C07D 401/12; A61K 31/4523; A61K 31/4545; C07D 211/66; A61K 45/06; A61K 51/048
USPC 424/1.65; 540/474; 514/326; 546/194; 514/318; 546/221; 514/327; 424/450

(57) ABSTRACT

The present invention provides compound conjugates of matrix metalloprotease inhibitors and linked metal chelators which are useful for imaging solid tumors and treating and diagnosing certain types of diseases such as cancer.

### CROSS-REFERENCES TO RELATED APPLICATIONS

This application claims the benefit of priority to U.S. Provisional Application Ser. No. 61/791,387, filed Mar. 15, 2013, the content of which is incorporated herein by reference in its entirety.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Most, if not all, solid tumors produce locally high concentrations of matrix metalloprotease (MMP) enzymes. These enzymes are either excreted to the surrounding matrix or are membrane bound on the tumor cell surface (A. Matter, Drug Discovery Today 2001; 6: 1005-1024). For example, Matrix Metalloproteinase-14 (MT1-MMP or MMP-14) is a membrane-associated protease implicated in a variety of tissue remodeling processes and a molecular hallmark of select metastatic cancers (M. D. Sternlicht, et al., Oncogene 2000; 19: 1102-13). Other MMPs, such as gelatinases (MMP-2 and MMP-9), are able to degrade type IV collagen, which is a major component of the basement membrane, and have been shown to be involved in tumor invasiveness, metastasis, and angiogenesis [L. A. Liotta, et al., Nature 1980; 284: 67-68.]. Increased expression of MMP-2, in particular, has been found in a variety of malignant tumors, including breast, lung, gastric, and esophageal carcinomas (S. Furumoto, K. Takashima, K. Kubota, T. Ido, R. Iwata, H. Fukuda, Nuclear Medicine and Biology 2003:30:119-125).

There is currently an urgent need for inhibitors of MMPs which are also useful as targeting agents as well as for molecular imaging. This need is due, in part, to the fact that MMP activity is directly linked to the metastatic potential of tumors and MMP activity is also strongly associated to angiogenesis and tumor growth. Both of these characteristics are critical with respect to evaluating the aggressiveness of a tumor in clinical imaging. Also, patients having tumors can be treated with an analogous radiotherapeutic agent if MMP expression is detected.

The present invention provides solutions to challenges outlined above as well as to many others. For example, the compounds and compositions described of the present invention have been discovered to be surprisingly useful as small molecule MMP inhibitors that target MMP-14 (MT1-MMP) and MMP-2 with high affinity and are therefore suitable imaging probes and/or radiotherapeutic agents for cancer.

### BRIEF SUMMARY OF THE INVENTION

In one aspect, the present invention provides a compound conjugate including a matrix metalloprotease inhibitor (MMPi) portion and a linked metal chelator portion.

In a second aspect, the present invention provides a method of diagnosing the presence of solid tumors in a patient. The method includes administering a compound conjugate including a matrix metalloprotease inhibitor (MMPi) portion and a linked metal chelator portion to a patient; acquiring a contrast image of the administered conjugate, or fragment thereof, in a patient or subject; analyzing said contrast image to determine the concentration of MMP enzymes; and; diagnosing the presence of solid tumors based on said determination of the concentration of MMP enzymes. With respect to this method, a locally high concentration of MMP enzymes indicates a diagnosis of solid tumors.

In a third aspect, the present invention provides a method for treating a patient having solid tumors. The method includes administering a compound conjugate including a matrix metalloprotease inhibitor (MMPi) portion and a linked metal chelator portion to a patient having solid tumors; thereby treating a patient having solid tumors.

### BRIEF DESCRIPTION OF THE DRAWINGS

1/11

FIG. 1 shows a plot which indicates the binding and internalization of ¹¹¹-In MP-3563, ¹¹¹-In MP-3590, and ¹¹¹-In MP-3591 to HT-1080 MT1 cells.

FIG. 2 shows a plot which indicates the binding and internalization of ¹¹¹-In MP-3563, ¹¹¹-In MP-3590, and ¹¹¹-In MP-3591 to HT-1080 MT1 cells.

2/11

FIG. 3 shows a plot indicating the binding of ¹¹¹-In MP-3590 to MCF-7-MT1 cells.

3/11

FIG. 4 shows a plot indicating the binding of ¹¹¹-In MP-3647 to MCF-7-MT1 cells.

FIG. 5 shows % tumor levels as a function of time (hr) and amount of cold MP-3590 blocking dose as determined by administering ¹¹¹-In MP-3590 in vivo to mice (biodistribution study in nude mice bearing HT1080 MT1 xenograft tumors).

4/11

FIG. 6 shows % tumor levels as a function of time (hr) and amount of cold blocking dose as determined by administering ¹¹¹In MP-3590 in vivo to mice (biodistribution study in nude mice bearing HT1080 MT1 xenograft tumors).

FIG. 7 shows ¹¹¹-In MP-3590 uptake as a function of time and cold MP-3590 blocking dose from an imaging study of nude mice bearing HT1080 MT1 xenograft tumors.

5/11

FIG. 8 shows the PK of MP-3563 and MP-3590 in Nude Mice with HT1080-MT1 Tumors.

FIG. 9 shows MMP-2 enzyme activity as a function of concentration of ARP100.

6/11

FIG. 10 shows MMP-2 enzyme activity as a function of concentration of Batimastat.

FIG. 11 shows MMP-2 enzyme activity as a function of concentration of MP3659.

FIG. 12 shows MMP-2 enzyme activity as a function of concentration of MP3661.

FIG. 13 shows MMP-14 enzyme activity as a function of concentration of ARP100.

FIG. 14 shows MMP-14 enzyme activity as a function of concentration of Batimastat.

9/11

FIG. 15 shows MMP-14 enzyme activity as a function of concentration of MP3659.

FIG. 16 shows MMP-14 enzyme activity as a function of concentration of MP3661.

FIG. 17 shows the binding of [⁶⁷Ga]-MP3661 and [⁶⁷Ga]-MP3618 to MCF-MT1 cells at 37° C. as a function of ligand concentration.

11/11

FIG. 18 shows SPECT/CT biodistribution of radiolabeled ligand (¹¹¹In-DTPA-Lys-Phenyl-O-Phenyl) for MT1 receptors in NSG mice bearing MCF7 tumors (MCF7 parental tumors and MCF7 tumors overexpressing MT1).

FIG. 19 shows SPECT/CT imaging of a radiolabeled ligand for MT1 receptors in mice bearing MCF 7 tumors. The images were collected 24 hours post dosing. The left image is for a MCF-7 parental xenograft (injected dose 480 while the right image is for a MCF-7 MT1+ xenograft (injected dose 466 μCi).

### DETAILED DESCRIPTION OF THE INVENTION

### I. General

The present invention sets forth compositions, e.g., compound conjugates of matrix metalloprotease inhibitors and linked metal chelators, which are useful for imaging solid tumors and treating and diagnosing certain types of diseases such as cancer.

### II. Definitions

As used herein, the term “conjugate” or “compound conjugate” refers generally to a molecule that includes a linking group. In some embodiments, a conjugate of the present invention has the formula: C-L-MMPi. In this definition, C represents a metal chelator, L represents a linking group, and MMPi represents an MMP inhibitor. Attachment of these components can be achieved through a functional group using a linking chemistry known in the art or by the methods set forth herein.

As used herein, the term “linking group” refers to part of a conjugate that links two components, e.g., an MMPi and a chelator. In some instances, the linking group used herein has the formula of

[chemical structure]

wherein A¹ indicates the point of attachment to the MMPi portion; and A² indicates the point of attachment to the metal chelator portion.

As used herein, the term “bifunctional” refers to a molecule that has at least two points to which another chemical can be attached.

As used herein, the term “chelator” refers to a compound or portion of a compound that coordinates a metal ion, e.g., Ca²⁺ or Zn²⁺. Chelators bond to metal ions via non-metallic ligands, e.g., alkyl-CO₂⁻. Chelators can be referred to as bidentate when two ligands, or chemical groups, are used to coordinate a metal ion. A common chelator is EDTA which is also known as ethylenediaminetetraacetic acid.

As used herein, the term “stealth agent” refers to a molecule that can modify the surface properties of a composition set forth herein. A stealth agent can prevent compositions from sticking to each other and to blood cells or vascular walls. Stealth agents for use in the present invention can include those generally well known in the art. In certain embodiments, a stealth agent can include “polyethylene glycol,” which is well known in the art and refers generally to an oligomer or polymer of ethylene oxide. Polyethylene glycol (PEG) can be linear or branched, wherein branched PEG molecules can have additional PEG molecules emanating from a central core and/or multiple PEG molecules can be grafted to the polymer backbone. PEG can include low or high molecular weight PEG, e.g., PEG500, PEG2000, PEG3400, PEG5000, PEG6000, PEG9000, PEG10000, PEG20000, or PEG50000 wherein the number, e.g., 500, indicates the average molecular weight. Other suitable stealth agents can include but are not limited to dendrimers, polyalkylene oxide, polyvinyl alcohol, polycarboxylate, polysaccharides, and/or hydroxyalkyl starch.

As used herein, the term “lipid” refers to lipid molecules that can include fats, waxes, sterols, cholesterol, fat-soluble vitamins, monoglycerides, diglycerides, phospholipids, sphingolipids, glycolipids, cationic or anionic lipids, derivatized lipids, and the like. Lipids can form micelles, monolayers, and bilayer membranes. In certain embodiments, the lipids can self-assemble into liposomes. In other embodiments, the lipids can coat a surface of a nanocarrier as a monolayer or a bilayer.

As used herein, the term “subject” or “patient” refers to any mammal, in particular human, at any stage of life.

As used herein, the terms “administer,” “administered,” or “administering” refers to methods of administering the compounds and compositions of the present invention. The compositions of the present invention can be administered in a variety of ways, including topically, parenterally, intravenously, intradermally, intramuscularly, colonically, rectally or intraperitoneally. Parenteral administration and intravenous administration are the preferred methods of administration. The compositions can also be administered as part of a pharmaceutical formulation.

As used herein, the terms “treating” or “treatment” of a condition, disease, disorder, or syndrome includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

As used herein, the term “formulation” refers to a mixture of components for administration to a subject. Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. The formulations of the present invention can be presented in unit-dose or multi-dose sealed containers, such as ampoules and vials. A composition described herein, alone or in combination with other suitable components, can be made into aerosol formulations (i.e., they can be “nebulized”) to be administered via inhalation through the mouth or the nose. Aerosol formulations can be placed into pressurized acceptable propellants, such as dichlorodifluoromethane, propane, nitrogen, and the like. Suitable formulations for rectal administration include, for example, suppositories, which comprise an effective amount of a composition described herein with a suppository base. Suitable suppository bases include natural or synthetic triglycerides or paraffin hydrocarbons. In addition, it is also possible to use gelatin rectal capsules which contain a combination of a composition, described herein, with a base, including, for example, liquid triglycerides, polyethylene glycols, and paraffin hydrocarbons. In certain embodiments, formulations can be administered topically or in the form of eye drops.

### III. Compounds and Compositions

In some embodiments, the present invention provides a compound conjugate including a matrix metalloprotease inhibitor (MMPi) portion and a linked metal chelator portion.

In some embodiments, the present invention provides a compound conjugate including a matrix metalloprotease inhibitor (MMPi) portion and a linked metal chelator portion wherein the MMPi is linked to the metal chelator portion by a bifunctional linker. A variety of bifunctional linking groups are useful in the compounds and compositions described herein. In general, the linking group should be one that does not interfere with the targeting properties of the MMPi component, and should be stable while in circulation (e.g., less than 20% hydrolysis or enzymatic cleavage separating the MMPi and metal chelator portions). The linker may have additional functional groups which can serve to increase solubility.

Suitable linkers are generally available from commercial sources such as Thermo Scientific's Pierce Protein Biology Products catalog (or can be modified from such readily available sources). The bifunctional linker will generally have an amine-reactive end for conjugation to the MMPi which may be selected from, but are not limited to, activated esters, imidoesters, anhydrides, isocyanates, isothiocyanates, acyl azides, epoxides, sulfonyl chlorides, carbonates, and aldhehydes (reductive amination). In addition to single amino acids for linking the MMPi to the chelator, peptide sequences may be utilized as well. Derivatized polyethylene glycol polymers also serve as acceptable linkers for this invention. Conversion of the MMPi's piperidine amine to a thiolated analog with Traut's reagent (2-iminothiolane) would allow conjugation to sulfhydryl-reactive linkers. Examples of linkers with sulfhydryl-reactive moieties include, but are not limited to, maleimide, haloacetyl, pyridyl disulfide, thiosulfonate, and vinyl sulfone. Chelators may be derivatized with functional groups for linker conjugation which may differ from the specific examples shown. Depending on the functionality on the chelator or MMPi, linkers may be homobifunctional or heterobifunctional. In addition, suitable modification of both the chelator and MMPi would allow for direct conjugation of the two pieces. For example, the copper-catalyzed azide-alkyne cycloaddition may be employed if either the MMPi or chelator has an azide and its counterpart has an alkyne. Further appropriate conjugation techniques may be found in Bioconjugation Techniques (Greg T. Hermanson, Academic Press: New York (1996)).

Some examples of suitable linkers are depicted below.

[chemical structure]

[chemical structure]

For the linkers described above, in some embodiments, A¹ indicates the point of attachment to the MMPi portion. In some embodiments, A² indicates the point of attachment to the metal chelator portion. The subscript ‘n’ is an integer, generally from 1-10, and in some embodiments, the subscript is 1, 2, 3, 4, 5, 6, 7, 8, 9 or 10.

In still other embodiments, the bifunctional linker has the following structure

[chemical structure]

wherein A¹ indicates the point of attachment to the MMPi portion and A² indicates the point of attachment to the metal chelator portion.

In some embodiments, the conjugate of the present invention has the following structure:

[chemical structure]

In some embodiments, R¹ is selected from the group consisting of O and S. In some embodiments, R² is selected from the group consisting of pyridyl and phenyl. In some embodiments, when R² is phenyl, said phenyl at R² is optionally substituted with 1-5 substituents selected from OH, OCH₃, OCF₃ or CH₃. In some embodiments, R³ is the linked metal chelator portion.

In some embodiments, R³ is selected from

[chemical structure]

wherein the wavy line indicates the point of attachment to the remainder of the conjugate.

In some embodiments, the present invention provides a conjugate, as described herein, having the following structure:

[chemical structure]

wherein R⁴ is selected from

[chemical structure]

and
wherein the wavy line indicates the point of attachment at R⁴.

In some embodiments, the present invention provides a conjugate, as described herein, having a structure selected from

[chemical structure]

In some embodiments, the present invention provides a conjugate a structure selected from

[chemical structure]

[chemical structure]

In some embodiments, the present invention provides a conjugate, as described herein, that includes a radionuclide.

In some embodiments, the present invention provides a conjugate, as described herein, that includes a radionuclide is selected from ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y, or ⁹⁰Y.

In some embodiments, the present invention provides a conjugate, as described herein, wherein the radionuclide is selected from ¹¹¹In or ^{99m}Tc.

In some embodiments, the present invention provides a conjugate, as described herein, including a lanthanide.

In some embodiments, the present invention provides a conjugate, as described herein, that also includes a lipid, a liposome, a micelle, a lipid-coated bubble, or a block copolymer micelle. In certain embodiments, the lipid is a phospholipid, glycolipid, sphingolipid, or cholesterol.

In some embodiments, the present invention provides a conjugate, as described herein, that includes a stealth agent. In certain embodiments, the stealth agent is poly(ethylene glycol).

In some embodiments, the present invention provides a conjugate, as described herein, that is formulated as a pharmaceutical composition that includes a conjugate that is described herein.

In some embodiments, MMPi refers to any matrix metalloproteinase inhibitor. In certain embodiments, MMPi is an inhibitor having the formula:

[chemical structure]

X¹ is a member selected from the group consisting of O and S. Y¹ is a member selected from the group consisting of pyridyl and phenyl, wherein said phenyl is optionally substituted with OH, OCH₃, OCF₃ and CH₃; and the wavy line indicates the point of attachment to a linking group and, or, a metal chelator.

In certain specific embodiments, MMPi is selected from:

[chemical structure]

In certain specific embodiments, MMPi is selected from:

[chemical structure]

A radioisotope can be incorporated into a composition or conjugate described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga.

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8,11-tetraazacyclotetradec-1-yl)methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

Other chelators which are useful with the compositions set forth herein include, but are not limited to, NOTA, NODAGA, and TRAP chelates (e.g., for Ga-67/68) and the inverted histidine chelate for Tc-99m carbonyl.

### IV. Methods of Making the Compounds and Compositions Described Herein

A variety of methods of making compounds set forth herein are taught in the Examples. Additional methods which are known to a skilled chemist may be used in conjunction with those taught herein.

### V. Methods for Diagnosing

In some embodiments, the present invention provides a method of diagnosing the presence of solid tumors in a patient. The method includes administering a compound conjugate comprising a matrix metalloprotease inhibitor (MMPi) portion and a linked metal chelator portion to a patient. The method also includes acquiring a contrast image of the administered conjugate, or fragment thereof, in the patient. The method also includes analyzing said contrast image to determine the concentration of MMP enzymes. The method also includes diagnosing the presence of solid tumors based on said determination of the concentration of MMP enzymes. In some methods, a locally high concentration of MMP enzymes indicates a diagnosis of solid tumors.

In some embodiments, the present invention provides a method of diagnosing or treating a disease or condition selected from cancer, carcinoma, sarcoma, leukemia, lymphoma, myeloma, central nervous system cancer, solid tumors, or mixed tumors or combinations thereof.

In some embodiments, the present invention provides a method for treating a patient having solid tumors. The method includes administering a compound conjugate including a matrix metalloprotease inhibitor (MMPi) portion and a linked metal chelator portion to a patient having solid tumors, with or without a therapeutic isotope; thereby treating a patient having solid tumors.

### VI. Methods of Treatment

The compositions set forth herein may be suitable for inhibiting matrix metalloproteinases. The compositions set forth herein may be suitable for imaging and diagnosing the presence of matrix metalloproteinases. In part because of this, the compositions which are described herein may be useful as part of a treatment regiment for a variety of diseases which are mediated, to some extent, by the activity of matrix metalloprotenases. For example, see Greenwald, et al., Inhibition of Matrix Metalloproteinases—Therapeutic Applications, Annals of the New York Academy of Science, Volume 878, 1999 which recites diseases which can be diagnosed or treated using the compositions set forth herein.

Therapeutic agents can be selected depending on the type of disease desired to be treated and incorporated with the compositions described herein. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include chemotherapeutic agents, such as alkylating agents, antimetabolites, anthracyclines, alkaloids, topoisomerase inhibitors, and other anticancer agents. In some embodiments, the agents can include antisense agents, microRNA, siRNA and/or shRNA agents.

### VII. Diagnostic Agents

A diagnostic agent may be used in the present invention and can include any diagnostic agent known in the art, as provided, for example, in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, a diagnostic agent can include chelators that bind, e.g., to metal ions to be used for a variety of diagnostic imaging techniques. Exemplary chelators include but are not limited to ethylenediaminetetraacetic acid (EDTA), [4-(1,4,8,11-tetraazacyclotetradec-1-yl)methyl]benzoic acid (CPTA), Cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7,10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope can be incorporated into some of the diagnostic agents described herein and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga.

In other embodiments, the diagnostic agents can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these. Other agents that can be used include, but are not limited to, for example, fluorescein, fluorescein-polyaspartic acid conjugates, fluorescein-polyglutamic acid conjugates, fluorescein-polyarginine conjugates, indocyanine green, indocyanine-dodecaaspartic acid conjugates, indocyanine-polyaspartic acid conjugates, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid.

One of ordinary skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents of the present invention will absorb or be excited by light in the ultraviolet (UV), visible, or infrared (IR) range of the electromagnetic spectrum. For imaging, dyes that absorb and emit in the near-IR (˜700-900 nm, e.g., indocyanines) are preferred. For topical visualization using an endoscopic method, any dyes absorbing in the visible range are suitable.

In some embodiments, the non-ionizing radiation employed in the process of the present invention can range in wavelength from about 350 nm to about 1200 nm. In one exemplary embodiment, the fluorescent agent can be excited by light having a wavelength in the blue range of the visible portion of the electromagnetic spectrum (from about 430 nm to about 500 nm) and emits at a wavelength in the green range of the visible portion of the electromagnetic spectrum (from about 520 nm to about 565 nm). For example, fluorescein dyes can be excited with light with a wavelength of about 488 nm and have an emission wavelength of about 520 nm. As another example, 3,6-diaminopyrazine-2,5-dicarboxylic acid can be excited with light having a wavelength of about 470 nm and fluoresces at a wavelength of about 532 nm. In another embodiment, the excitation and emission wavelengths of the optical agent may fall in the near-infrared range of the electromagnetic spectrum. For example, indocyanine dyes, such as indocyanine green, can be excited with light with a wavelength of about 780 nm and have an emission wavelength of about 830 nm.

In yet other embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to Gadopentetic acid, Gadoteric acid, Gadodiamide, Gadolinium, Gadoteridol, Mangafodipir, Gadoversetamide, Ferric ammonium citrate, Gadobenic acid, Gadobutrol, or Gadoxetic acid. Superparamagnetic agents can include but are not limited to superparamagnetic iron oxide and Ferristene. In certain embodiments, the diagnostic agents can include x-ray contrast agents as provided, for example, in the following references: H. S Thomsen, R.N. Muller and R. F. Mattrey, Eds., Trends in Contrast Media, (Berlin: Springer-Verlag, 1999); P. Dawson, D. Cosgrove and R. Grainger, Eds., Textbook of Contrast Media (ISIS Medical Media 1999); Torchilin, V. P., Curr. Pharm. Biotech. 1:183-215 (2000); Bogdanov, A. A. et al., Adv. Drug Del. Rev. 37:279-293 (1999); Sachse, A. et al., Investigative Radiology 32(1):44-50 (1997). Examples of x-ray contrast agents include, without limitation, iopamidol, iomeprol, iohexyl, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexyl, iopentol, ioversol, iobitridol, iodixanol, iotrolan and iosimenol.

Similar to therapeutic agents described above, the diagnostic agents can be associated with the conjugates herein in a variety of ways, including for example being tethered to the MMPi portion through a chelate.

### VIII. Pharmaceutical Compositions

The invention provides pharmaceutical compositions of the compounds described herein. The pharmaceutical compositions of the present invention encompass compositions made by admixing a compound of the present invention and a pharmaceutically acceptable carrier and/or excipient or diluent. Such compositions are suitable for pharmaceutical use in an animal or human.

The pharmaceutical compositions of the present invention comprise a compound described herein, or a pharmaceutically acceptable salt thereof, as an active ingredient and a pharmaceutically acceptable carrier and/or excipient or diluent. A pharmaceutical composition may optionally contain other therapeutic ingredients.

The compounds of the present invention can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

Compositions for topical administration include, but are not limited to, ointments, creams, lotions, solutions, pastes, gels, sticks, liposomes, nanoparticles, patches, bandages and wound dressings. In certain embodiments, the topical formulation comprises a penetration enhancer.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

Compositions for systemic administration include, but are not limited to, dry powder compositions consisting of the powder of a compound described herein, or a salt thereof, and the powder of a suitable carrier and/or excipient. The compositions for systemic administration can be represented by, but not limited to, tablets, capsules, pills, syrups, solutions, suspensions, films and suppository.

With respect to formulations with respect to any variety of routes of administration, methods and formulations for the administration of drugs are disclosed in Remington: The Science and Practice of Pharmacy, 21st Edition, Lippincott Williams & Wilkins Eds., 2005; and in Ansel's Pharmaceutical Dosage Forms and Drug Delivery Systems, 8^{th} Edition. Lippincott Williams & Wilkins Eds., 2005, which are herein incorporated as reference.

### IX. Administration

In some embodiments, the present invention can include a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized compositions.

The composition set forth herein, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which includes an effective amount of a packaged composition with a suppository base. Suitable suppository bases include natural or synthetic triglycerides or paraffin hydrocarbons. In addition, it is also possible to use gelatin rectal capsules which contain a combination of the composition of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of compositions can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a pharmaceutical composition including a compound described herein. The unit dosage form can be a packaged preparation, the package containing discrete quantities of preparation. The composition can, if desired, also contain other compatible therapeutic agents.

In therapeutic use for the treatment of cancer, the compositions of the invention including a therapeutic and/or diagnostic agent utilized in the pharmaceutical compositions of the present invention can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular composition in a particular patient. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. For convenience, the total daily dosage may be divided and administered in portions during the day, if desired.

In some embodiments, the compositions of the present invention may be used to diagnose a disease, disorder, and/or condition. In some embodiments, the compositions can be used to diagnose a cancerous condition in a subject, such as lung cancer, breast cancer, pancreatic cancer, prostate cancer, cervical cancer, ovarian cancer, colon cancer, liver cancer, esophageal cancer, and the like. In some embodiments, methods of diagnosing a disease state may involve the use of the compositions to physically detect and/or locate a tumor within the body of a subject. For example, tumors can be related to cancers that sufficiently express (e.g., on the cell surface or in the vasculature) a receptor that is being targeted by a targeting agent of a composition of the present invention. In some embodiments, the compositions set forth herein can also be used to diagnose diseases other than cancer, such as proliferative diseases, cardiovascular diseases, gastrointestinal diseases, genitourinary disease, neurological diseases, musculoskeletal diseases, hematological diseases, inflammatory diseases, autoimmune diseases, rheumatoid arthritis and the like.

As disclosed herein, the compositions of the invention can include a diagnostic agent that has intrinsically detectable properties. In detecting the diagnostic agent in a subject, the compositions of the invention can be administered to a subject. The subject can then be imaged using a technique for imaging the diagnostic agent, such as single photon emission computed tomography (SPECT), magnetic resonance imaging (MRI), optical imaging, positron emission tomography (PET), computed tomography (CT), x-ray imaging, gamma ray imaging, and the like. Any of the imaging techniques described herein may be used in combination with other imaging techniques. In some embodiments, the incorporation of a radioisotope for imaging in a particle allows in vivo tracking of the compositions in a subject. For example, the biodistribution and/or elimination of the compositions can be measured and optionally be used to alter the treatment of patient. For example, more or less of the compositions may be needed to optimize treatment and/or diagnosis of the patient.

### X. Kits

The present invention also provides kits for administering the compositions of the invention to a subject for treating and/or diagnosing a disease state. Such kits typically include two or more components necessary for treating and/or diagnosing the disease state, such as a cancerous condition. Components can include compositions of the present invention, reagents, containers and/or equipment. In some embodiments, a container within a kit may contain a composition including a radiopharmaceutical that is radiolabeled before use. The kits can further include any of the reaction components or buffers necessary for administering the compositions. Moreover, the compositions can be in lyophilized form and then reconstituted prior to administration.

In certain embodiments, the kits of the present invention can include packaging assemblies that can include one or more components used for treating and/or diagnosing the disease state of a patient. For example, a packaging assembly may include a container that houses at least one of the compositions as described herein. A separate container may include other excipients or agents that can be mixed with the compositions prior to administration to a patient. In some embodiments, a physician may select and match certain components and/or packaging assemblies depending on the treatment or diagnosis needed for a particular patient.

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

In some embodiments, the present invention includes a fluorescent reporter prototype that targets the cellular active MT1-MMP enzyme alone. This reporter may include a liposome loaded with a fluorochrome and functionalized with a polyethylene glycol chain spacer linked to an inhibitory hydroxamate warhead. This composition may be useful to visualize the trafficking of MT1-MMP through the cell compartment. This composition may be useful to quantify the femtomolar range amounts of the cell surface-associated active MT1-MMP enzyme in multiple cancer cell types, including breast carcinoma, fibrosarcoma and melanoma.

### XI. Examples

### Abbreviations

ACN, acetonitrile; mL, milliliters; HOBT, hydroxybenzotriazole; LC/MS, liquid chromatography mass spectrum; Cbz, benzyloxycarbonyl, DCM, dichloromethane, DMF, dimethylformamide; DMSO, dimethyl sulfoxide; EDC, 1-(3-Dimethylaminopropyl)-3-ethylcarbodiimide HCl; H, hexane; RBF, round bottom flask; rt, ambient temperature; h, hour(s); TLC, thin layer chromatography; TEA, triethylamine; TFA, trifluoroacetic acid, HRMS, high resolution mass spectrum; Boc, tert-butyloxycarbonyl.

The following compounds are referenced below:

| Compound | Structure |
|---|---|
| MP 3647 | [chemical structure] |
| MP 3563 | [chemical structure] |
| MP 3577 | [chemical structure] |
| MP 3590 | [chemical structure] |
| MP 3591 | [chemical structure] |
| MP 3593 | [chemical structure] |
| MP-3659 | [chemical structure] |
| MP 3661 | [chemical structure] |
| MP-3618 | [chemical structure] |

### Example 1

Specific binding of ¹¹¹In MP 3590 to HT-1080 MT1 cells was readily apparent and saturable with MP-3590, but not with other compounds, at doses tested. Furthermore, internalization of MP-3590 was appreciable by HT-1080 MT1 cells under these conditions (See 1/11 FIG. 1 & FIG. 2).

### Example 2

Specific binding of ¹¹¹In MP 3590 to MCF-7-MT1 cells was observed. Binding was saturable and in an acid resistant manner to transfected cells @ 4° C. (Kd=6.8 nM). At 37° C., a significant portion of the bound ligand was internalized (See 2/11 FIG. 3). For the data shown in 2/11

FIG. 3, 24 wells of MCF7 cells (1.7×10⁵ cells/well) or MCF7-MT1 cells (2.4×10⁵ cells/well) were incubated at 37° C. for 1 h (top panel) or 4° C. for 2 h (lower panel) with the indicated concentration of radioligand, then washed three times with binding buffer (DMEM/F 12/0.1% BSA) at pH 7.4 (total binding) or pH 2.5 (intracellular binding), lysed and counted. MP-3590 bound specifically and saturably and in an acid sensitive manner to transfected cells at 4° C. (Kd=6.8 nM). At 37° C., significant amounts of bound ligand were internalized. as ligand was protected from acid elution from the cells). At 50 nM starting concentration, 4.4×10⁶ CPM were applied to the cells. Radioligand was prepared at 200 μCi/mmol and 6 μM stock.

### Example 3

Specific, saturable binding of ¹¹¹In MP 3647 to MCF-7-MT1 cells was observed. In addition, a significant portion of the bound ligand was internalized at 37° C. (see 3/11 FIG. 4). For the data in 3/11

FIG. 4, MCF-7 human tumor cells (1×10⁵/well) or MCF-7-MT1 transfected cells (4.5×10⁵/well) were incubated at 4° C. or 37° C./5% CO₂ for 2 h with 0.5 ml DMEM/F12+0.1% BSA+25 mM HEPES+radioligand (¹¹¹In-MP-3647, 8.51×10⁶ CPM/well). 24-well clusters were used for these experiments. Cells were placed at 4° C., media was aspirated, and cells were washed three times with 0.5 mL of the buffer described above at pH 7.4 (total binding) or at pH 2.5 (to remove extracellular radioligand). Cells were lysed with SDS/0.1 N NaOH and counted. Individual determinations are shown. Binding was specific and saturable to MT-1 expressing cells, with little apparent internalization of the radioligand seen by the cells following binding. At 4° C. on MCF-7 MT-1 cells, the apparent Kd for radioligand binding is 0.98 nM. In a separate functional assay, inhibition of MMP-2 activity (0.7 nM EC50), and MMP-14 activity (25.2 nM EC50) was obtained with this compound.

### Example 4

¹¹¹In MP-3590 was evaluated in vivo in imaging and biodistribution studies, and specific binding was observed in all cases as evidenced by the reduction in binding in the presence of cold ligand.

¹¹¹In MP-3590 was administered i.v. to nude mice bearing HT-1080 tumors over expressing MT1 (MMP-14). Each animal received 5 μCi indium labeled compound (0.04 μg) and 0 or 9.6 μg excess cold material to block binding of the radiolabeled compound. The addition of unlabeled competitor inhibited tumor accumulation of radiolabeled MP3590 (See FIG. 5).

### Example 5

Biodistribution Study: 111In MP-3590 was administered i.v. to nude mice bearing HT-1080 tumors over expressing MT1 (MMP-14). Each animal received 5 μCi indium labeled compound (0.04 μg) and 0, 0.4, and 100 μg excess cold material to block binding of the radiolabeled compound. Each compound cleared rapidly from mice. The addition of unlabeled competitor inhibited tumor accumulation of radiolabeled MP3590 (See 4/11 FIG. 6). MP-3590 exhibited dose-dependent inhibition in tumor accumulation with addition of unlabeled competitor.

### Example 6

Imaging Study: ¹¹¹In MP-3590 was administered i.v. to nude mice bearing HT-1080 tumors over expressing MT1 (MMP-14). Each animal received ca. 400 μCi indium labeled compound. Two mice were injected IV with low specific activity MP3590 (0.7 Ci/mmol) and two mice were injected IV with high specific activity MP3590 (2000 Ci/mmol). Mice were imaged ˜1, 4 and 24 hours post dose. Tumor data given below. Once again, the addition of unlabeled competitor inhibited tumor accumulation of radiolabeled MP3590 (See FIG. 7).

### Example 7

The inhibition potencies of MP-3590 and MP-3647 against activated MMP-2, and MMP-14 were assayed using a synthetic fluorogenic substrate. Recombinant human MMPs were activated 1-2 hours before use and then pre-incubated with test compounds. An aliquot of the substrate was added and fluorescence changes were monitored using a microplate analyzer. Inhibition curves were plotted (as a function of inhibitor concentration) and IC₅₀ values were calculated by nonlinear regression analysis. Batimastat and ARP100 are used as controls during screening. Batimastat is a potent, broad spectrum matrix metalloprotease (MMP) inhibitor which inhibits both MMP-2 and MMP-14 at low nM levels, and ARP100 is a selective MMP-2 inhibitor (and a poor inhibitor for MMP-14). The ARP100 and Batimastat were from TOCRIS Bioscience, Bristol, UK (now part of R&D Systems).

| Compound | rhMMP2 | rhMMP14 |
|---|---|---|
| MP-3590 | IC₅₀ = 0.8 | IC₅₀ = 118 |
| MP-3647 | IC₅₀ = 0.7 | IC₅₀ = 25.2 |

MMP-2 and MMP-14 enzyme activities were measured using recombinant human proteins, substrate and protocols provided by R & D Systems, Minneapolis, Minn. For the assays, 25 μl, of 5× test samples and 50 μL of activated recombinant human protein were combined in 96 black well plates at ambient temperature, and 50 μL of substrate (Mca-Lys-Pro-Leu-Gly-Leu-Dpa-Ala-Arg-NH₂, R&D ES010) was then added to start the reaction. (All components were prepared at concentrations and in appropriate buffers as listed below.) The plates were then read at 320 nm excitation/405 nm emission using the kinetic mode of a fluorescence plate reader. A substrate blank was subtracted from duplicate determinations and IC₅₀ values from six point dose response curves were calculated using GraphPad Prism software, La Jolla, Calif.

For the MMP-14 enzyme assay, 40 μg/mL of rhMMP-14 (R&D 918-MP) was activated with 0.86 μg/mL rhFurin (R&D 1503-SE) in activation buffer (50 mM Tris, 1 mM CaCl₂, 0.05% (v/v) Brij-35, pH 9) and incubated at 37° C. for 1.5 hours. The activated protein was then diluted to 1.24 μg/mL in assay buffer (50 mM Tris, 3 mM CaCl₂, 1 μM ZnCl₂, pH 8.5), for use in the assay. The substrate was used at 20 μM in assay buffer.

For the MMP-2 enzyme assay, 100 μg/mL of rhMMP2 (R&D 902-MP) was activated with 1 mM APMA (p-aminophenylmercuric acetate) in assay buffer (50 mM Tris, 10 mM CaCl₂, 150 mM NaCl, 0.05% (v/v) Brij-35, pH 7.5) and incubated at 37° C. for 1 hour. The activated protein was then diluted to 248 ng/mL in assay buffer for use in the assay. The substrate was used at 25 μM in assay buffer.

FIG. 9-FIG. 12 show results for MMP-2 enzyme activity for ARP100, Batimastat, MP-3659 and MP-3661, respectively.

FIG. 13-FIG. 16 show results for MMP-14 enzyme activity for ARP100, Batimastat, MP-3659 and MP-3661, respectively.

### Example 8

### Synthesis Procedures

### Preparation of MMPi-Radiolabeled Compounds

### Preparation of MP-3590

### Step 1

[chemical structure]

A 100-mL RBF equipped with magnetic stir bar was charged with 4-methyl 4-((4-phenoxyphenyl)sulfonyl)piperidine-4-carboxylate (1.1 g, 2.67 mmol), Cbz-OSu (0.73 g, 2.94 mmol), and triethylamine (0.8 g, 8.0 mmol). LC/MS after 1 hour showed complete reaction with M+H=510 g/mol. The reaction was partitioned between ethyl acetate and saturated aqueous sodium bicarbonate. The organic layer was washed with 10% aq KHSO₄, dried, and concentrated to thick syrup that turned into white dry foam upon high vacuum drying. The product was dried overnight to afford 1.29 g (95% yield) of the Cbz ester that was used in Step 2.

### Step 2

[chemical structure]

A 250-mL RBF was charged with 1-benzyl-4-methyl 4-((4-phenoxyphenyl)-sulfonyl)piperidine-1,4-dicarboxylate (1.75 g, 3.39 mmol) and potassium hydroxide (0.57 g, 10.2 mmol) in 30 mL ethanol/7.5 mL water. The reaction mixture was stirred at 50° C. with LC/MS monitoring. LC/MS analysis indicated ˜80% conversion after 1 hour and approximately 90% conversion after 2 hours with trace impurities appearing. The solution was concentrated to 1/4 volume and partitioned between ethyl acetate and 10% aqueous citric acid. The organic layer was washed with brine, dried, and concentrated in vacuo. The product was vacuum-dried overnight to yield 1.6 g (95% yield) white solid that was used in Step 3.

### Step 3

[chemical structure]

A 50-mL RBF equipped with magnetic stir bar was charged with 1-((benzyloxy)carbonyl)-4-((4-phenoxyphenyl)sulfonyl)piperidine-4-carboxylic acid (1.48 g, 2.99 mmol), OTHP-hydroxylamine (0.49 g, 4.18 mmol), EDC (0.8 g, 4.18 mmol), HOBt (0.64 g, 4.18 mmol), and triethylamine (1.25 mL, 8.96 mmol) in 30 mL DMF. The reaction mixture was stirred at room temperature overnight. The reaction was concentrated in vacuo and partitioned between ethyl acetate and saturated aqueous sodium bicarbonate. The organics were washed with 10% aqueous citric acid, brine, dried, and concentrated in vacuo. This material was vacuum-dried overnight to afford 1.50 g (85%) of dry white foam. The sample was analyzed by direct infusion MS, which indicated >90% product with some trace impurities. HRMS (theoretical) M+H=595.2108 g/mol. HRMS (observed) M+H=595.2109 g/mol.

### Step 4

[chemical structure]

A 20-mL RBF equipped with magnetic stir bar was charged with benzyl 4-((4-phenoxyphenyl)sulfonyl)-4-(((tetrahydro-2H-pyran-2-yl)oxy)carbamoyl)piperidine-1-carboxylate (1.5 g) and 100 mg wet Degussa 5% Palladium on Carbon in 45 mL of methanol. The reaction mixture was purged with argon for 5 minutes. Hydrogen was then bubbled over the solution for 1 hour. MS analysis (direct infusion) at this point indicated that the reaction was complete. The crude was filtered through Celite, and the Celite was washed with 40 mL additional methanol. The methanol solution was concentrated in vacuo to 1.2 g white solid that was vacuum-dried for 4 hours to yield 1.1 g of product that was used in Step 5. HRMS (observed) M+H=451.1737 g/mol.

### Step 5

[chemical structure]

A 50-mL RBF equipped with magnetic stir bar was charged with 4-((4-phenoxyphenyl)sulfonyl)-4-(((tetrahydro-2H-pyran-2yl)oxy)carbamoyl)piperidine (330 mg, 0.72 mmol), acid (286 mg, 0.75 mmol), EDC (172 mg, 0.9 mmol), HOBt (165 mg, 1.1 mmol), and triethylamine (218 mg, 2.15 mmol) in 10 mL dry DMF. The reaction mixture was stirred at room temperature overnight. The DMF was removed, and the reaction residue was partitioned between ethyl acetate and saturated aqueous sodium bicarbonate. The organic layer was washed with brine, dried, concentrated, and vacuum-dried to afford 585 mg (97% yield) crude white foam that was used in Step 6. HRMS (theoretical) M+Na=845.3402 g/mol. HRMS (observed) M+H=845.3406 g/mol.

### Step 6

[chemical structure]

A 100-mL RBF was charged with 585 mg crude product from Step 6, 88 mg 5% wet Palladium on Carbon (Degussa) in 45 mL methanol. The reaction mixture was purged with argon for ˜5 minutes, then hydrogen was slowly bubbled over the solution. LC/MS analysis after 1 hour showed ˜50-60% conversion to product with a M+H=689 g/mol.

LC/MS analysis indicated about 75% conversion after three hours and about 90% conversion after four hours. The mixture was left to react for an additional hour. After a 5-minute argon purge, the reaction mixture was filtered through Celite. The reaction was concentrated, and volatiles were chased 2× with dichloromethane. The white foam/solid was vacuum dried overnight to afford 492 mg (87%) white solid. The product is referred to as MP-3656.

### Step 7

[chemical structure]

A 50-mL RBF was charged with the tri-t-butyl DTPA acid (100 mg, 0.29 mmol), EDC (42 mg, 0.22 mmol), and HOBt (34 mg, 0.22 mmol) in 4 mL of DMF. After 15 minutes, triethylamine (59 mg, 0.58 mmol) was added and the mixture was stirred for an additional 15 min. Next, amine (from Step 6, 100 mg, 0.145 mmol) in 3 mL DMF was added, and the reaction was stirred at room temperature overnight. LC/MS indicated there was a 60:40 ratio of desired product to dimer. The reaction was concentrated and dried under high vacuum overnight. RP-HPLC purification was accomplished on C-18 using a flow rate of 30 mL/min and a 15-95% acetonitrile gradient over 20 minutes. The product-containing fractions were combined and lyophilized to afford 40 mg of product.

### Step 8

[chemical structure]

The 100-mL RBF used to lyophilize the sample from Step 7 was charged with 5 mL TFA and tumbled for 3½ hours. By LC/MS, the reaction was complete. It was concentrated, taken up in water and acetonitrile, and lyophilized to afford 36 mg of MP-3590.

### Preparation of MP-3659

### Step 1

[chemical structure]

A 50-mL plastic centrifuge tube was charged with bromoacetic Wang resin (750 mg 0.75 mmol), the protected histidine (748 mg, 2.2 eq, 1.65 mmol), and Hunig's base (388 mg 3.0 mmol). This reaction mixture was put on the shaker overnight. The resin was washed with 10 mL DMF, twice with 15 mL dichloromethane, twice with 20 mL ether and dried covered over for approximately forty-eight hours.

### Step 2

[chemical structure]

The resin product from Step 1 was transferred to a shaker flask and swelled with 10 mL DCM for 10 minutes. The DCM was filtered off and discarded. The resin was treated with 5×10 mL 1 TFA in DCM (2 minutes shaking each time) and filtered into a flask containing 4 mL 10% pyridine in MeOH. The resin was than washed with 2×20 mL DCM, 2×20 mL MeOH, and 1×20 mL DCM. The combined filtrates were concentrated in vacuo and partitioned between ethyl acetate and 10% aqueous citric acid. The organic layer was washed with brine, dried, and concentrated to afford crude oil. LC/MS shows product with a trace of bis adduct.

### Step 3

[chemical structure]

A 10-mL RBF equipped with magnetic stir bar was charged with amine (see Step 6 for MP-3590, 226 mg, 0.33 mmol), protected histidine acid (166 mg, 0.33 mmol), EDC (76 mg, 0.39 mmol), HOBt (63 mg, 0.39 mmol), and 100 mg (0.98 mmol) triethylamine in 5 mL DMF. The reaction mixture was stirred at room temperature overnight. The reaction mixture was partitioned between ethyl acetate and saturated aqueous bicarbonate. The organic layer was washed with 10% aq.citric acid. Brine was added to separate the emulsion. The organic layer was separated, dried, and concentrated in vacuo. TLC (20% ethyl acetate-Hexane) showed no movement. LC/MS demonstrated a big TIC around 2.5 where HOBT usually comes and product at 4.9 minutes. The residue was treated with chloroform and filtered to remove insolubles before drying over Na₂SO₄ and concentrating to give 450 mg oil. This was used as is in Step 4.

### Step 4

[chemical structure]

The crude product from Step 3 was treated with TFA (9.5 mL), triethylsilane (0.25 mL), and water (0.25 mL). After an hour, the starting material was gone. Peaks were observed at 3.9, 4.0, and 4.1 with main M+H=826 g/mol. After 4 hr, the reaction was concentrated in vacuo and vacuum-dried overnight to obtain ˜350 mg semi-solid. Four mL of 0.1% TFA in water and 2 mL of acetonitrile were added and tumbled at 30° C. and then centrifuged at 3500 rpm for 9 minutes. The solution was then filtered through a 0.45 μM Millipore filter. Purification was performed by RP-HPLC using a 10-95% acetonitrile gradient over 20 minutes with a 30 mL/min flow rate. The product-containing fractions were concentrated to ½ volume and lyophilized over the weekend. Final weight was 18.1 mg. The product was referred to as MP-3659.

### Preparation of MP-3647

### Step 1

[chemical structure]

A 5-mL RBF equipped with magnetic stir bar was charged with amine (see Step 6 for MP-3590, 205 mg, 0.3 mmol), 1,4,7,10-Tetraazacyclododecane-1,4,7,10-tetraacetic acid mono (N-hydroxysuccinimide) ester (DOTA NHS ester, 243 mg, 0.3 mmol), and triethylamine (60 mg, (0.6 mmol) in 10 mL dichloromethane. The reaction mixture was stirred at room temperature overnight. LC/MS showed product as small M+H/big M+Na. Additional 40 mL of dichloromethane (DCM) were added, and the organic layer was washed with 50 mL satd. aq. bicarbonate and 10 mL brine. The organic layer was dried and concentrated in vacuo. LC/MS indicated that the desired product was obtained. The material was vacuum-dried for 5 hours. After drying, 240 mg of white waxy solid were obtained.

### Step 2

[chemical structure]

The 100-mL RBF containing the product of the previous step (240 mg) was charged with 5 mL of 4N HCl-dioxane and tumbled at 30° C. with LC/MS monitoring. LC/MS after 4 hours showed no starting material but also no product. Two peaks with earlier retention times but higher mass than desired product were observed. After 7 hours, it appeared that product was starting to form at the 3.6 min. peak which was much larger than before. It also appeared that the 969/970 peak might well be the mono t-butyl product as the M+Na. The reaction was continued overnight. LC/MS showed almost exclusively the 3.6 min. peak with an M+H=891 g/mol. The reaction was concentrated in vacuo. The crude solid was slurried up in cold ether, filtered, and washed with cold ether to afford 200 mg.

The sample showed some free DOTA at solvent front and desired product at 9 minute retention time. HRMS obs M+H=891.3869 g/mol. HRMS simulated M+H=891.3917 g/mol. The compound was registered as MP-3647.

### Preparation of MP-3661

### Step 1

[chemical structure]

A 50-mL RBF equipped with magnetic stir bar was charged with amine (prepared as described in Step 6 for MP-3590, 100 mg, 0.145 mmol), S-2-(4-isothiocyanatobenzyl)-1,4,7-triazacyclononane-1,4,7-triacetic acid (81 mg, 0.145 mmol), and triethylamine (94 mg, 0.73 mmol) in 4 mL DMF. The reaction went pale yellow and homogenous. The reaction was covered in foil and stirred at RT in the dark. LC/MS after 2 hours showed traces of the isocyanate. LC/MS after 4 hours showed small trace of starting material. The reaction mixture was concentrated to a crude mixture before dissolving in 2 mL acetonitrile and 6 mL 0.1% TFA in water. Afterward, the solution was filtered through a 0.45 μM Millipore filter. Purification was accomplished by RP-HPLC using a 10-95% acetonitrile gradient over 20 minutes with a 30 mL/min. flow rate. Product came off at 12 minutes (˜76% ACN). The product-containing fraction were concentrated to ¼ volume for lyophilization after freezing which provided 94 mg of the free hydroxamate tris-TFA salt.

### Step 2

[chemical structure]

The crude tris-TFA salt from Step 1 was slurried in 5 mL of 4N HCl/dioxane and tumbled for 15 minutes. LC/MS indicated new peak at 3.7 min. The sample was given to analytical for HRMS. HRMS (negative ion) Theo M−H=953.3616 g/mol. Observed M−H=953.3698 g/mol. After concentration in vacuo and vacuum-drying, there were 83 mg of product that was referred to as MP-3661.

### Preparation of MP-3577

### Step 1

[chemical structure]

A 100-mL RBF flask equipped with magnetic stir bar was charged with Boc-protected amine (prepared as described in Step 6 for MP-3590, 440 mg, 0.64 mmol) and 10 mL anhydrous DMF, and the reaction flask was cooled in an ice-bath. To the cooled flask was added triethylamine (259 mg, 2.56 mmol) followed by ENPy2 acid (208 mg, 0.77 mmol) (AN-4282-72), HOBt (117 mg, 0.77 mmol) and EDC (147 mg, 0.77 mmol). The reaction mixture was allowed to warm-up to room temperature and stirred overnight under inert atmosphere. After 21 h, the reaction was complete and DMF was removed using a rotary evaporator. The concentrated reaction mixture was dissolved in 150 mL of ethyl acetate and washed with 35 mL of saturated sodium bicarbonate followed by 40 mL of brine. The organic layer was separated and dried over Na₂SO₄ and concentrated in vacuo. The crude product was dried under high vacuum overnight. The crude yield was 690 mg (brown sticky solid), which was used in the next step without purification.

### Step 2

[chemical structure]

A 100-mL RBF equipped with magnetic stir bar was charged with 602 mg crude ENPy derivative and dissolved in 10 mL of anhydrous DCM. The reaction flask was cooled in an ice bath, and ˜5 mL of TFA was added. The reaction was stirred for 4 hours (monitoring the reaction by LC/MS every hour) until LC-MS showed completion of reaction. Reaction mixture was concentrated in vacuo and dried under high vacuum overnight. The yield of concentrated product was 1.47 g (brown oil, might have some residual TFA). This product was dissolved in about 6 mL of acetonitrile and purified using reverse phase chromatography.

Gradient Used: 5% B/0 min, 5% B/2 min, 95% B/15 min, 95% B/16 min, 5% B/16.5 min and 5% B/20 min. [Where A: 25 mM ammonium acetate in water with 5% Acetonitrile and B: Acetonitrile/Isopropanol (1:1)]. Column: Waters XBridge Preo C18, 5 um OBD, 30×150 mm column. Injections made: 100 uL, 500 uL and (750 mL×8). The desired product eluted around 10 minutes. Pure fractions were consolidated, and acetonitrile was removed from the solution. The remaining solution was lyophilized overnight. Yield of the dried product was 220 mg (white powder) and referred to as MP-3577.

### Preparation of MP-3563

### Step 1

[chemical structure]

A 50-mL RBF equipped with magnetic stir bar was charged with the mono-DTPA acid (231 mg, 0.31 mmol), EDC (70 mg, 0.37 mmol), and HOBt (70 mg, 0.46 mmol) in 3 mL of DMF. The amine (187 mg, 0.31 mmol) was dissolved in 5 mL DMF and added, followed by triethylamine (93 mg, 0.9 mmol). The reaction mixture was stirred at room temperature overnight. The reaction mixture was concentrated in vacuo and partitioned between ethyl acetate and satd. aq bicarbonate. The organic layer was separated and washed with 10% aq. KHSO₄, brine, and dried. The organic layer was then filtered, concentrated, and vacuum-dried overnight to afford 200 mg of crude material. LC/MS indicated mostly product with an impurity with M+H=1031 g/mol. This material was deprotected and purified in Step 2.

### Step 2

[chemical structure]

A 50-mL RBF was charged with the 200 mg crude product from Step 1, and TFA (about 8 mL) and stirred at room temperature for 5 hours. The TFA was removed in vacuo, chased 2× with ether, and vacuum-dried to an off-white solid overnight. LC/MS showed product as broad peak and some impurities. The observed M+H=953.3560 g/mol. The material was purified with ammonium acetate/IPA-acetonitrile on a C8 column. The product-containing fractions were concentrated and lyophilized overnight. A second purification was done using 90% water-10% acetonitrile with 0.1% TEA to afford 30 mg of product referred to as MP-3563.

### Preparation of MP-3593

### Step 1

[chemical structure]

A 25-mL RBF was charged with amine (60 mg, 0.1 mmol), tri-t-butyl DTPA (55 mg, 0.1 mmol), EDC (23 mg, 0.12 mmol), HOBt (18 mg, 0.12 mmol), and triethylamine (30 mg, 0.3 mmol) in 4 mL DMF. The reaction was stirred under argon overnight. LC/MS analysis showed the formation of two products. The first eluting peak was the desired product. The reaction mixture was concentrated in vacuo and the crude product was purified using reverse phase chromatography. Yield of the desired product=10.8 mg. The product was referred to as MP 3591.

### Step 2

[chemical structure]

A 25-mL RBF was charged with 10.8 mg starting material (from Step 1) and was dissolved in 2 mL neat TFA and stirred under argon for 3 hr. LC/MS analysis showed that, after 3.5 hour, the reaction was complete. Water was added to the reaction mixture. After freezing, the material was lyophilized for approximately forty-eight hours. The yield of dry product was 11.2 mg and was referred to as MP-3593.

### Preparation of MP-3618

### Step 1

[chemical structure]

A 100-mL RBF equipped with a magnetic stir bar was charged with amine (140 mg, 0.23 mmol), acid (260 mg, 0.46 mmol), EDC (66 mg, 0.35 mmol), HOBt (55 mg, 0.35 mmol), and triethylamine (93 mg, 0.93 mmol) in 8 mL DMF and stirred at room temperature overnight. The DMF was removed, and the 1/2 mL remaining was diluted with 5-6 mL acetonitrile and filtered through a 0.45 uM Millipore filter. LC/MS indicated ˜1:1 peak of desired to dimer. RP-HPLC using a 15-100% acetonitrile gradient over 20 minutes with a 30 mL flow rate. The product-containing fractions were concentrated until cloudy (˜15 mL), and about 8 mL water and 8 mL fresh acetonitrile were added before freezing and lyophilizing. 50 mg of product were obtained.

### Step 2

[chemical structure]

The lyophilized product from Step 1 was treated with 2 mL TFA in the 150 mL lyophilization jar and was permitted to sit for 4 hours at room temperature. 30 mL of water were added before freezing and lyophilizing overnight. 2 mL TFA was added to the lyophilized product, and it was permitted to sit for 4 hours then diluted with 20 mL of water before freezing and lyophilizing over the weekend. The dry material was dissolved in 10 mL of water and lyophilized overnight in a tared 4 dram vial. After drying over P₂O₅/NaOH, 36 mg of the tetra-TFA salt were obtained. This product was referred to as MP-3618.

Procedure for In-111 labeling of MMPi-DTPA conjugates (MP-3590, MP-3593, MP-3563, MP-3618)

In a 1.5 mL Eppendorf tube, 30 μL 0.1 M NaOAc was added, pH 5.5, to 10 μL 50 μM DTPA-MMPi compound, 5×10⁻¹⁰ mol, and 30 μL ¹¹¹InCl₃ (ca. 500 μCi) in 0.05HCl. The mixture was incubated for 15 minutes at room temperature. The mixtures were analyzed by HPLC using a C-18 HPLC column with water:acetonitrile:0.1% TFA mobile phase. All radiochemical purities were >90%.

The specific HPLC conditions include the following: Column: Waters Nova-Pak C18, 4 μm, 3.9×150 mm, s/n 11573113714038. Mobile Phase A: 95% H₂O/5% ACN, 0.1% TFA. Mobile Phase B: 95% ACN/5% H₂O, 0.1% TFA. 0-5 min 0% B. 5-25 min 0% B-90% B. 26 min 90% B-0% B. 26-30 min 0% B. Flow rate 1 mL/min. Temperature ambient

Procedure for In-111 labeling or Ga-67 labeling of MMPi-DOTA and MMPi-NOTA conjugates (MP-3647, MP-3661)

In a 1.5 mL Eppendorf tube, 30 μL 0.1 M NaOAc was added, pH 5.5, to 10 tit 50 μM DOTA-MMPi or NOTA-MMPi compound, 5×10⁻¹⁰ mol, and 30 μL ¹¹¹InCl₃ (ca. 500 μCi) or ⁶⁷GaCl₃ (ca. 500 μCi). The mixture was incubated for 20 minutes at 95° C. The mixtures were analyzed by HPLC using a C-18 HPLC Column with water:acetonitrile:0.1% TFA mobile phase. All radiochemical purities were >80%. Specific HPLC conditions are the same as above.

### Procedure for Tc-99m labeling of MP-3577 and MP-3659

### Step 1. Kit Preparation of [^{99m}Tc(CO)₃(OH₂)₃]⁺

To a 10 mL sealed tubing vial containing the following lyophilized formulation: 8.5 mg sodium tartrate.2H₂O, 2.85 mg sodium tetraborate.10H₂O, 7.15 mg of sodium carbonate, and 4.5 mg sodium boranocarbonate, 1 mL of ^{99m}TcO₄⁻ from a commercial generator (20-100 mCi) were added. The vial was placed in a boiling water bath for 15 minutes. Quality control effected by reverse phase HPLC (C-18 column with a 0.05 M TEAP; pH=2.25/methanol gradient) shows >95% radiochemical purity (retention time=3.9 min).

The specific HPLC conditions are:
Column: Vydac C18 (250×4.6 mm, 5μ) (serial number=218TP54)
Mobile phase:
   A: TEAP 0.05 M, pH 2.25 (0.5 l water+7 mL Triethylamine+H3PO4 85%, about two Pasteur pipettes, which will give a pH of ca. 3 and adjust pH to 2.25 with phosphoric acid. Finally fill to 1 l with water and filter).
   B: MeOH 100%
Gradient: 0 to 3 min 100% A
   3 to 6 min from 100 to 75% A
   6 to 9 min from 75 to 66% A
   9 to 20 min from 34 to 100% B
   20 to 27 min 100% B
   27 to 30 from 100% B to 100% A

### Step 2: Formation of [^{99m}Tc(CO)₃(MP-3577)] or [^{99m}Tc(CO)₃(MP-3659)]

In the second step, 235 μL of 1 N HCl was added to the prepared kit to bring the solution pH value to 4 and decompose any residual sodium boranocarbonate. 1 N NaOH was carefully added to adjust the solution pH value to 5.0-5.5. Next, to 500 μL of the [^{99m}Tc(CO)₃(OH₂)₃]⁺ solution, 5-10 μL of MMPi conjugate (1 mg/mL water) was added. The resulting solution was heated at 100° C. for 20 minutes. Radiochemical purity values are >90%. 26 min 90% B-0% B. 26-30 min 0% B. Flow rate 1 mL/min. Temperature ambient.

### Example 9

MP3563 (pyridyl) and MP3590 (phenyl-O-phenyl) were administered iv to nude mice bearing HT-1080 tumors overexpressing MT1 (MMP-14). Each animal received 5 μCi indium labeled compound (0.04 μg) and 0, 0.4, and 100 μg excess cold material to block binding of the radiolabeled compound. Each compound cleared rapidly from mice. See 5/11 FIG. 8.

### Example 10

### Competitive Binding Assay

### Experimental Details:

Twelve-well clusters of subconfluent MCF7-MT1 cells were rinsed and incubated at 37° C./5% CO₂ for 1 h with 0.5 mL/well in duplicate DMEM/F12+25 mM HEPES+0.1% BSA (binding media) containing 1 nM radioligand ⁶⁷Ga-MP-3661 (0.92 μM stock; 186 μCi) and the indicated amount of unlabeled test competitors. Following the binding interval, media was aspirated and cells washed 3 times with ice-cold binding media. Monolayers were then lysed in 0.5 mL/well 1% SDS/0.1N NaOH and counted. Triplicate assessments of binding of radioligand with no added competitor are also shown.

### Results:

Specific binding was observed since cold MP-3661 efficiently competed with the hot binding with an IC50 of <1 nM. Some competition was observed with cold MP-3618 around the 0.1-1 μM level perhaps due to non-specific lipophilic interactions with the cell membrane.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound conjugate comprising a matrix metalloprotease inhibitor (MMPi) portion and a linked metal chelator portion.

2. The conjugate of claim 1, wherein the MMPi is linked to the metal chelator portion by a bifunctional linker.

3. The conjugate of claim 2, wherein the bifunctional linker has the following structure [chemical structure]
wherein A¹ indicates the point of attachment to the MMPi portion; and
A² indicates the point of attachment to the metal chelator portion.

4. The conjugate of claim 1, having the following structure: [chemical structure]
wherein R¹ is selected from the group consisting of O and S;
wherein R² is selected from the group consisting of pyridyl and phenyl;
wherein when R² is phenyl, said phenyl at R² is optionally substituted with 1 to 5 substituents selected from the group consisting of OH, OCH₃, OCF₃ and CH₃; and
wherein R³ is the linked metal chelator portion.

5. The conjugate of claim 4, wherein R³ is selected from the group consisting of [chemical structure]
and
wherein the wavy line indicates the point of attachment to the remainder of the conjugate.

6. The conjugate of claim 4, having the following structure: [chemical structure]
wherein R⁴ is selected from the group consisting of [chemical structure]
and
wherein the wavy line indicates the point of attachment at R⁴.

7. The conjugate of claim 4, having a structure selected from the group consisting of

[chemical structure]

[chemical structure]

8. The conjugate of claim 4, having a structure selected from the group consisting of

[chemical structure]

9. The conjugate of claim 1, and further comprising a radionuclide.

10. The conjugate of claim 9, wherein said radionuclide is selected from the group consisting of ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶re, ¹⁸⁸Re, ⁴⁷Sc, ¹⁵³Sm, ⁸⁹Sr, ^{99m}Tc, ⁸⁸Y and ⁹⁰Y.

11. The conjugate of claim 9, wherein said radionuclide is selected from the group consisting of ¹¹¹In and ^{99m}Tc.

12. The conjugate of claim 1, and further comprising a lanthanide.

13. The conjugate of claim 1, and further comprising a lipid, a liposome, a micelle, a lipid-coated bubble, or a block copolymer micelle.

14. The conjugate of claim 13, wherein said lipid is a phospholipid, glycolipid, sphingolipid, or cholesterol.

15. The conjugate of claim 1, and further comprising a stealth agent.

16. The conjugate of claim 15, wherein said stealth agent is poly(ethylene glycol).

17. A pharmaceutical composition comprising a conjugate of claim 1.

18. A method of diagnosing the presence of solid tumors in a patient, the method comprising the following steps:
administering a compound conjugate comprising a matrix metalloprotease inhibitor (MMPi) portion and a linked metal chelator portion to a patient;
acquiring a contrast image of the administered conjugate, or fragment thereof, in the patient;
analyzing said contrast image to determine the concentration of MMP enzymes; and;
diagnosing the presence of solid tumors based on said determination of the concentration of MMP enzymes;
wherein a locally high concentration of MMP enzymes indicates a diagnosis of solid tumors.

19. The method of claim 18, wherein the solid tumors are associated with a disease or condition selected from the group consisting of cancer, carcinoma, sarcoma, leukemia, lymphoma, myeloma, central nervous system cancer, solid tumors, and mixed tumors.

20. A method for treating a patient having solid tumors, the method comprising:
administering a compound conjugate comprising a matrix metalloprotease inhibitor (MMPi) portion and a linked metal chelator portion to a patient having solid tumors;
thereby treating a patient having solid tumors.
